SEQUENCE LISTING (4 sequences)

SEQ ID NO: 1 - DNA, 2061 bp, Triticum aestivum L.
atggacgcctccgccggctcgtcgccgccgcggcactcacagggggacgcgccgaggcgcggcgggccgcaccgcgggaagggccccgcggtggagatccggcagggagaggacgacttcatgttcgcgcaggataccttcccggccctcccggacttcccttgcctctcctcgccgtcgagctccaccttctcctcctcctcgtcttccaactcctccagcgccttcgctgccccagcgggagcgggcgggcgcggggccgagggggcgcgcggcgagccgtccgagcctgcagcggccggggacgggatggacgacctctccgacatcgaccacctgctcgacttcgcgtccatcaacgacgacgtcccctgggacgacgagccgctcttccccgacgtcgggatgatgctggaggatgtcatctccgagcagcagctccagcagcctccggcgggccacggcacggccgggagaacggcgccgcatgcggcctctggtggaggagaggatgccttcatgggtggcggcggcacggggagcgcggcggacgacctgccgctgttcttcatggagtggctcaagaacaaccgcgactgcatctcggccgaggacctccgcagcatccgcctccgtcgatccaccatcgaggccgcggccgcgcgcctcggtggggggcgccagggcaccatgcagctgctcaagctcatcctcacctgggtgcagaaccaccacctgcagaagaagcgcccccgcgtcggcgccatggatcaggaggcgccgccggcaggaggccagctccctagccccggcgcaaaccccggctacgaattccccgcggagacgggtgccgccgctgccacatcatggatgccctaccaggccttctcgccaactggatcctacggcggcggggcgatctacccattccagcagggctgcagcactagcagcgtggccgtgagcagccagccgttctccccgccggcggcgcccgacatgcacgccggggcctggcccctgcagtacgcggcgttcgtcccagctggagccacatccgcaggcactcaaacatacccgatgccgccgccgggggccgtgccgcagccgttcgcggcccccggattcgccgggcagttcccgcagcggatggagccggcggcgaccagggaggcccggaagaagcggatggcgaggcagcggcgcttgtcgtgcctgcagcagcagcggagccagcagctgaatctgagccagatccaaaccggcggcttccctcaagagcaatccccccgcgcggcgcactcggcgccggtcacgccgccgtcgtccggctggggaggcctctgggcgcaacaggccgtccagggccagctcatggtccaggtcccgaatccgctgtcgacgaagtccaattcctcaaggcagaagcagcaaaaaccctcgccggacgcagcagcgaggccgccctccggcggcgccgccacgccgcatcgcccggggcaggcgtcggcttccaataagcagcggcagcagggtgcgaggacgccggcggcggcgccggcggcaggagacaagaacctgcggttcctgctgcagaaggtgctcaagcagagcgacgtcggaaccctcggccgcatcgtgctccccaaagaagcggagactcacctgccggagctcaagacgggggacggcatctcgatccccattgaggacatcggcacatctcaggtctggagcatgcggtaccgattttggcccaacaacaagagcagaatgtatcttctagagaacactggtgactttgttcggtccaatgagctgcaggagggtgatttcatcgtgctttactctgatgtcaagtcaggcaaatatctgatacgcggcgtgaaggtaagagctcaacaggatctagccaagcacaagaatgccagtccagagaaaggtggggcgtccgacgtgaaggcgggcggagaagacggtggttgcaaagagaagtctccgcacggtgtccggcgatctcgccaggaagccagctccatgaaccagatggccgtgagcatc

SEQ ID NO: 2 - protein, 687 aa, Artificial Sequence
  Description of Artificial Sequence Polypeptide sequence deduced from the polynucleotide sequence of SEQ ID No1.
MDASAGSSPPRHSQGDAPRRGGPHRGKGPAVEIRQGEDDFMFAQDTFPALPDFPCLSSPSSSTFSSSSSSNSSSAFAAPAGAGGRGAEGARGEPSEPAAAGDGMDDLSDIDHLLDFASINDDVPWDDEPLFPDVGMMLEDVISEQQLQQPPAGHGTAGRTAPHAASGGGEDAFMGGGGTGSAADDLPLFFMEWLKNNRDCISAEDLRSIRLRRSTIEAAAARLGGGRQGTMQLLKLILTWVQNHHLQKKRPRVGAMDQEAPPAGGQLPSPGANPGYEFPAETGAAAATSWMPYQAFSPTGSYGGGAIYPFQQGCSTSSVAVSSQPFSPPAAPDMHAGAWPLQYAAFVPAGATSAGTQTYPMPPPGAVPQPFAAPGFAGQFPQRMEPAATREARKKRMARQRRLSCLQQQRSQQLNLSQIQTGGFPQEQSPRAAHSAPVTPPSSGWGGLWAQQAVQGQLMVQVPNPLSTKSNSSRQKQQKPSPDAAARPPSGGAATPHRPGQASASNKQRQQGARTPAAAPAAGDKNLRFLLQKVLKQSDVGTLGRIVLPKEAETHLPELKTGDGISIPIEDIGTSQVWSMRYRFWPNNKSRMYLLENTGDFVRSNELQEGDFIVLYSDVKSGKYLIRGVKVRAQQDLAKHKNASPEKGGASDVKAGGEDGGCKEKSPHGVRRSRQEASSMNQMAVSI

SEQ ID NO: 3 - DNA, 20 bp, Artificial Sequence
  Description of Artificial Sequence Forward primer used to amplify the sequence of the gene of the invention.
tgcatggacgcctccgccgg

SEQ ID NO: 4 - DNA, 20 bp, Artificial Sequence
  Description of Artificial Sequence Reverse primer used to amplify the sequence of the gene of the invention.
tcagatgctcacsgccatct

(19) United States
(12) Patent Application Publication
(10) Pub. No.: US 2002/0148008 A1
(43) Pub. Date: Oct. 10, 2002

(54) Gene which regulates the dormancy of wheat seed

(76) Inventors: Shingo Nakamura, Iwate (JP); Katsumi Mori, Iwate (JP)

Correspondence Address: FINNEGAN, HENDERSON, FARABOW, GARRETT &, DUNNER LLP, 1300 I STREET, NW, WASHINGTON, DC, 20005, US

(21) Appl. No.: 09/820,346

(22) Filed: Mar. 29, 2001

(30) Foreign Application Priority Data
Nov. 17, 2000 (JP) 350363/2000

(51) Int. Cl. C12N 15/82; C12N 15/29; C12P 21/02

(52) U.S. Cl. 800/290; 800/286; 536/23.6; 530/370

(57) ABSTRACT

The present invention provides an isolated gene which regulates the dormancy of wheat seed, an antisense DNA to said gene, an isolated protein having activity which alters the degree of dormancy of wheat seed, a recombinant vector containing the gene, a transformant containing the vector; and a method for producing the protein.

### FIELD OF THE INVENTION

The present invention relates to an isolated gene which regulates the dormancy (resistance to preharvest sprouting) of wheat seed, an antisense DNA to said gene, an isolated protein having activity which alters the degree of wheat seed dormancy, a recombinant vector containing the gene, a transformant containing the vector, and a method for producing the protein.

### BACKGROUND OF THE INVENTION

VP1 has been isolated so far as one gene which is a cause of Viviporus of corn, and elucidated to be a transcriptional regulatory gene essential for formation of seed dormancy (McCarty et al., 1991). Furthermore, ABI3 gene isolated around the same time as a causal gene of abscisic acid-insensitive mutation of Arabidopsis seed, has been revealed to be a homologous gene of VP1 (Giraudat et al., 1992). Based on these studies, VP1 gene has been found to be a transcriptional factor which acts in the abscisic acid signaling system of a seed, playing a key role in formation of seed dormancy.

As seen from the above, at least one gene which plays a key role in the formation of seed dormancy is known to exist in some kinds of plants, however, it is still an open question as to what kind of gene action causes differences in the degree of wheat seed dormancy.

### SUMMARY OF THE INVENTION

The objectives of the present invention are to elucidate what kind of gene action causes differences in the degree of the wheat seed dormancy, and to provide a gene which regulates dormancy.

The inventors of the present invention have studied extensively and intensively to solve the above problems and have now found that the expression level of a certain gene exhibits correlation with the degree of dormancy in seeds of wheat varieties having different degrees of dormancy, and in wheat seeds in which dormancy has been disrupted by long-term storage. The present invention has been led to completion based on the above findings.

(i) Accordingly, the present invention relates to an isolated gene encoding a protein selected from the group consisting of the following (a) and (b):

(a) a protein comprising the amino acid sequence of SEQ ID NO: 2;

(b) a protein which comprises the amino acid sequence having deletions, substitutions or additions of one a plurality of amino acids in the amino acid sequence of SEQ ID NO: 2, and has activity which alters the degree of dormancy of wheat seed.

(ii) Furthermore, the invention relates to an antisense DNA to the gene of (i).

(iii) Still further, the present invention relates to an isolated protein selected from the group consisting of the following (a) and (b):

(a) a protein comprising the amino acid sequence of SEQ ID NO: 2;

(b) a protein which comprises the amino acid sequence having deletions, substitutions or additions of one or a plurality of amino acids in the amino acid sequence of SEQ ID NO: 2, and has activity which alters the degree of dormancy of wheat seed.

(iv) In addition, the invention relates to a recombinant vector comprising the gene of (i).

(v) The invention also relates to a recombinant vector comprising the antisense DNA of

(vi) Further, the invention relates to a transformant comprising the recombinant vector of (iv).

(vii) Further, the invention relates to a transformant comprising the recombinant vector of (v).

(viii) The invention also relates to a method for producing a protein having activity which alters the degree of dormancy of wheat seed comprising culturing the transformant of (vi) and recovering the protein from the resultant culture.

(ix) Further, the invention relates to a method for producing a protein having activity which alters the degree of dormancy of wheat seed comprising culturing the transformant of (vii) and recovering the protein from the resultant culture.

This specification includes part or all of the contents as disclosed in the specification and/or drawings of Japanese Patent Application No.2000-350363, which is a priority document of the present application.

### BRIEF DESCRIPTION OF THE DRAWINGS

FIG. 1 shows the results of comparison between a putative amino acid sequence encoded by a gene of the invention (SEQ ID NO: 2) and a putative amino acid sequence encoded by VP1 gene which is known in relation to different types of plants. Herein, Ta VP1 shows a putative amino acid sequence from a sequence of a gene of the invention presumed to be wheat VP1 gene, and AfVP1, Os and ZmVP1 show putative amino acid sequences from a sequence of VP1 gene which is known in relation to cat grass, rice and field corn, respectively

FIG. 2 shows the results obtained by performing Northern blot using DNA of a gene of the invention as a probe on mRNA from a seed of wheat varieties having various degrees of the dormancy and abscisic acid-sensitivity.

### DETAILED DESCRIPTION OF THE INVENTION

The present invention will be described in detail below.

An isolated gene of the present invention is characterized in that it encodes a protein selected from the group consisting of the following (a) and (b):

(a) a protein comprising the amino acid sequence of SEQ ID NO: 2;

(b) a protein which comprises the amino acid sequence having deletions, substitutions or additions of one or a plurality of amino acids in the amino acid sequence of SEQ ID NO: 2 and has activity which alters the degree of dormancy of wheat seed.

An isolated gene which has the nucleotide sequence of SEQ ID NO: 1 can be exemplified as a gene of the invention.

Among the above-mentioned genes, an gene encoding a protein comprising the amino acid sequence of SEQ ID NO: 2 can be obtained, for example, from a wheat according to a method which will be described in Example 1 hereinafter

Further, as shown in SEQ ID NO: 1, a gene in which the nucleotide sequence is once determined can be obtained, for example, by designing and synthesizing appropriate primers based on these nucleotide sequences, thereby performing PCR.

An isolated gene encoding a protein which comprises the amino acid sequence having deletions, substitutions or additions of one or a plurality of amino acids in the amino acid sequence of SEQ ID NO: 2 can be obtained according to common techniques used at the time when this application is filed, for example, site-directed mutagenesis method (Zoller et al., Nucleic Acids Res. 10 6478-6500, 1982) by means of modifying a sequence of a gene encoding a protein comprising the amino acid sequence of SEQ ID NO: 2. In this connection, “one or a plurality of amino acids” used herein is normally one or more amino acids, but is not limited as long as activity which alters the degree of dormancy of wheat seed is maintained in the protein where an amino acid sequence is modified by the above-mentioned method.

Furthermore, “a protein having activity which alters the degree of dormancy of wheat seed” means a protein which enhances dormancy (i.e. properties which prevent sprouting) by enhancing (improving) the resistance to dehydration in a seed where said protein is produced in the embryo thereof.

Furthermore, the term “isolated” used herein, means altered “by the hand of man” from the natural state. If an “isolated” composition or substance occurs in nature, it has been changed or removed from its original environment, or both.

By introducing a gene of the invention into varieties of wheat to regulate the expression level thereof, the degree of abscisic acid-sensitivity of seeds in those varieties can be altered, thereby the degree of dormancy of said seed can be changed. Herein, introduction of a gene can be carried out according to a method known to those skilled in the art for example, by introducing a gene of the invention into a protoplast of a plant from the wheat variety of interest, and reproducing.

Still further, an antisense DNA of the invention is characterized to be the antisense DNA to the above-described gene.

“Antisense DNA” used herein means DNA where the expression thereof functions to inhibit the expression of the above-mentioned gene, for example, DNA or fragment thereof which is complementary to the sequence of the gene in the above. Moreover, as long as it has the inhibiting function described above, the antisense DNA of the invention is not limited to one which is completely complementary to the above-mentioned gene, but can include, for example, partially mismatched sequence.

Accordingly, introducing the antisense DNA of the invention into various wheat varieties to inhibit the expression of a gene of the invention therein enables regulation of the dormancy of tie seed,

An isolated protein of the invention is characterized to be selected from the group consisting of the following (a) and (b):

(a) a protein comprising the amino acid sequence of SEQ ID NO: 2;

(b) a protein which comprises the amino acid sequence having deletions, substitutions or additions of one or a plurality of amino acids in the amino acid sequence of SEQ ID NO: 2, and has activity which alters the degree of dormancy of wheat seed.

Among proteins of the invention, a protein shown in an amino acid sequence of SEQ ID NO: 2 can be obtained, for example, by converting each series of 3 bases in the nucleotide sequence of SEQ ID NO: 1 by one amino acid which is encoded (or determined) by the combination of the bases (i.e. codon) by using an analysis software for a nucleic acid and amino acid sequence (Genetyx, software development, Tokyo) as described in EXAMPLE 3 hereinafter. Further, as shown in SEQ ID NO: 2 for instance, a protein of the invention in which an amino acid sequence thereof is once determined can be prepared based on the sequence according to a method known to those skilled in the art for example, a method where each amino acid is polymerized chemically to synthesize a protein peptide synthesis method) Still further, in the case where a protein of the invention is encoded by the gene of the invention in the above, a protein of the invention can be obtained for example by producing a recombinant vector including an isolated gene of the invention to introduce into a proper host cell, followed by culturing or raising the resulting transformant in a medium to recover the protein from the culture or the resultant. A recombinant vector, host cell, medium, each procedure and conditions used herein can be selected appropriately from those known to people skilled in the art.

“Deletions, substitutions or additions of one or a plurality of amino acids” in the amino acid sequence of SEQ ID NO: 2 can be performed according to common techniques used at the time when this application is fled, for example, site-directed mutagenesis method (Zoller et al., Nucleic Acids Res. 10 6478-6500, 1982) by means of modifying a sequence of a gene (e.g. a nucleotide sequence of SEQ ID NO: 1) which encodes a protein comprising the amino acid sequence of SEQ ID NO: 2.

In this connection, terms “one or a plurality of amino acids” and “a protein having activity which alters the degree of dormancy of wheat seed” used herein have the same meaning as is described in the section of an isolated gene of the invention.

### DESCRIPTION OF THE PREFERRED EMBODIMENTS

Embodiments of the invention will be further described below by means of illustration, but it is not intended that the scope of the invention is limited to them.

### EXAMPLE 1

The Confirmation of Seed Dormancy

Seeds obtained from 4 varieties of wheat (Zenkoujikomugi, Minaminokomugi, Sanin 1 and Tozan 18) and seeds artificially treated to decrease dormancy (Minaminokomugi, Sanin 1) were soaked individually in 5% (W/V) Benlate T (DuPont, USA) for 10 minutes, then sterilized by washing with water to be laid in petri dish. 8 ml of distilled water was added to them respectively, followed by culturing at 22° C. for 5 days to determine a sprouting rate (the number of sprouting seeds in the total number of tested seeds is indicated by %).

The results are shown in Table 1 below.

TABLE 1
The correlation between dormancy and sprouting rate of wheat

| | | | | Minaminokomugi (the | Sanin 1 | |
|---|---|---|---|---|---|---|
| | | | | dormancy has been | (the dormancy has | |
| | Zenkbujikomugi | Minaminokomugi | Sanin 1 | disrupted) | been disrupted) | Tozan 18 |
| Dormancy | strong | strong | weak | — | — | weak |
| Sprouting rate (%) | 30 | 0 | 70 | 100 | 100 | 100 |
| (22 ° C., 5 days) | | | | | | |

As seen from the results of table 1, a test sample of wheat “Zenkoujikomugi” and “Minaminokomugi” which has been said to have strong dormancy (resistance to preharvest sprouting) in general exhibits a low sprouting rate of 30% and 0% respectively, and a test sample of wheat; “Sanin 1” and “Tozan 18” which has been said to have weak dormancy in general exhibits a high sprouting rate of 70% and 100%, respectively. Accordingly, a wheat seed sample of each variety used in the examples hereinafter was confirmed to have natural dormancy. Additionally, both of the test samples, “Minaminokomugi” and “Sanin 1” which are artificially treated to decrease dormancy exhibited an extremely high sprouting rate of 100%, which confirmed that they had been correctly treated to disrupt dormancy as in the above.

### EXAMPLE 2

Analysis of Abscisic Acid-Sensitivity of a Seed Embryo in Each Variety

Next, seeds obtained from 3 varieties Minaminokomugi, Sanin 1 and Tozan 18) and seeds in which dormancy has been disrupted (Minaminokomugi, Sanin 1) were soaked individually in 5% (W/V) Benlate T(DuPont, USA) for 10 minutes, then sterilized by washing with distilled water. The grains of these wheat seeds were cut in halves to be laid in pet dish. A section where 8 mL of abscisic acid (ABA) solution was added and a section where 8 mL of distilled water was added were produced for each seed, followed by culturing at 22° C. for 6 days to determine the sprouting rate and the degree of sprout stretching.

The results of seeds of varieties having strong/weak dormancy and seeds in which dormancy has been disrupted by long-term storage are shown in Table 2 below,

TABLE 2
Fluctuation of the sprouting rate and the degree of sprout stretching in 5 kinds of wheat by the
abscisic acid treatment

| | | | | | | Minaminokomugi | Sanin 1 |
|---|---|---|---|---|---|---|---|
| | | | | | | (the dormancy has | (the dormancy has |
| | | | Minaminokomugi | Sanin 1 | Tozan 18 | been disrupted | been disrupted |
| Dormancy of seeds | | | strong | weak | weak | — | — |
| Seeds cut | Sprouting | + 10 μM | 0 | 100 | 100 | 70 | 100 |
| in halves | Rate (%) | ABA | | | | | |
| | | H₂O | 90 | 90 | 100 | 90 | 90 |
| | Length of | + 10 μM | 0 | 2.96 | 4.65 | 2.37 | 4.50 |
| | a | ABA | | (0.20) | (0.80) | (0.48) | (0.53) |
| | sprout(m | H₂O | 56 | 73 | 64 | 52 | 74 |
| | m) | | (6.6) | (1.9) | (3.0) | (6.5) | (2.3) |
| | (Standard | | | | | | |
| | error) | | | | | | |

As seen from the results of table 2, it was found that a seed of “Minaminokomugi” with strong dormancy had a strong abscisic acid-sensitivity, and seeds of “Sanin 1” and “Tozan 18” with weak dormancy, and seeds of “Minaminokomugi” and “Sanin 1” in which dormancy has been disrupted had weak abscisic acid-sensitivity or insensitivity. Taking into account the fact that a causal gene of mutation with abscisic acid-insensitivity in Arabidopsis was a homologous gene of field corn VP1 (described hereinbefore), a wheat of each variety, “Minaminokomugi” etc. is thought to have VP1 gene which affects the abscisic acid-sensitivity,

### EXAMPLE 3

Decoding of a Nucleotide Sequence of cDNA Presumed to be Wheat VP1 Gene

First of all, whole RNA was isolated from a mature embryo of a seed of wheat variety “Minaminokomugi” using Trizole agent (Gibco Lifetech Oriental, U.S.A). mRNA was then extracted from the whole RNA using a Latex beads kit (Takara). Using the obtained mRNA as a template, cDNA was prepared with Poly T (Gibco Lifetech Oriental, U.S.A.) as primers using reverse transcriptase and a cDNA synthesis kit (both products were made by Gibco Lifetech Oriental, U.S.A.).

EcoRI adaptor (Gibco Lifetech Oriental, U.S.A.) was then added to the 5′ end and 3′ end of said cDNA respectively, followed by subcloning in a plasmid vector pBluescript IISK+(Staratagene), thereby producing a cDNA library of a mature wheat embryo.

Furthermore, by using a sequence known to be specifically preserved in a sequence of corn VP1 gene as primers, PCR was performed with LA PCR kit (Takara) to obtain a full-length clone of a gene presumed to be wheat VP1 gene from said cDNA library. To be more specific, first of all, 1 μL of cDNA sample solution containing the fill-length clone (2.5 ngDNA/μL), 5 μL of 10×LA PCR buffer (Mg 25 mM). 8 μL of dNTP mixture (2.5 mM each), 0.5 μL of forward primer (20 pmol/μL), 0.5 μL of reverse primer (20 pmol/μL), 0.5 μL of Takara LA Taq and 34.5 μL of sterilized water were poured into a micro tube with capacities of 0.5 mL, and they were mixed to prepare a reaction solution. The solution was then set in GeneAmp PCR system 9700 (PE Applied Biosystems, USA), and 30 cycles with each temperature profile of 30 sec at 96° C. 30 sec at 60 and 150 sec at 72 were performed to amplify DNA of a putative gene of wheat VP1. Sequences of primers used were as follows.

| | | |
|---|---|---|
| (Primer) | | |
| Forward primer: | 5′-TGCATGGACGCCTCCGCCGG-3′ | (SEQ ID NO:3) |
| Reverse primer: | 5′-TCAGATGCTCAC(G/C)GCCATCT-3′ | (SEQ ID NO:4) |

The amplified DNA was cloned in pT7 Blue T Plasmid Novagene, U.S.A.).

A nucleotide sequence of a plasmid which contains desired DNA fragment was then determined based on dideoxy method using Dye Terminator Cycle Sequencing FS Ready Reaction kit (PE Biosystems Japan, Mode1310 type). The obtained nucleotide sequence is represented by SEQ ID NO: 1. This nucleotide sequence was further analyzed using an analysis software for a nucleic acid and amino acid sequence (Genetyx, software development Tokyo). A sequence of 5′ side and 3′ side were determined by performing 5′ RACE and 3′ RACE respectively with an obtained internal sequence.

From the result, a gene of the invention is thought to consist of 2061 bases (SEQ ID NO: 1), encoding a protein comprising 687 amino acids (the amino acid sequence is shown in SEQ ID NO: 2).

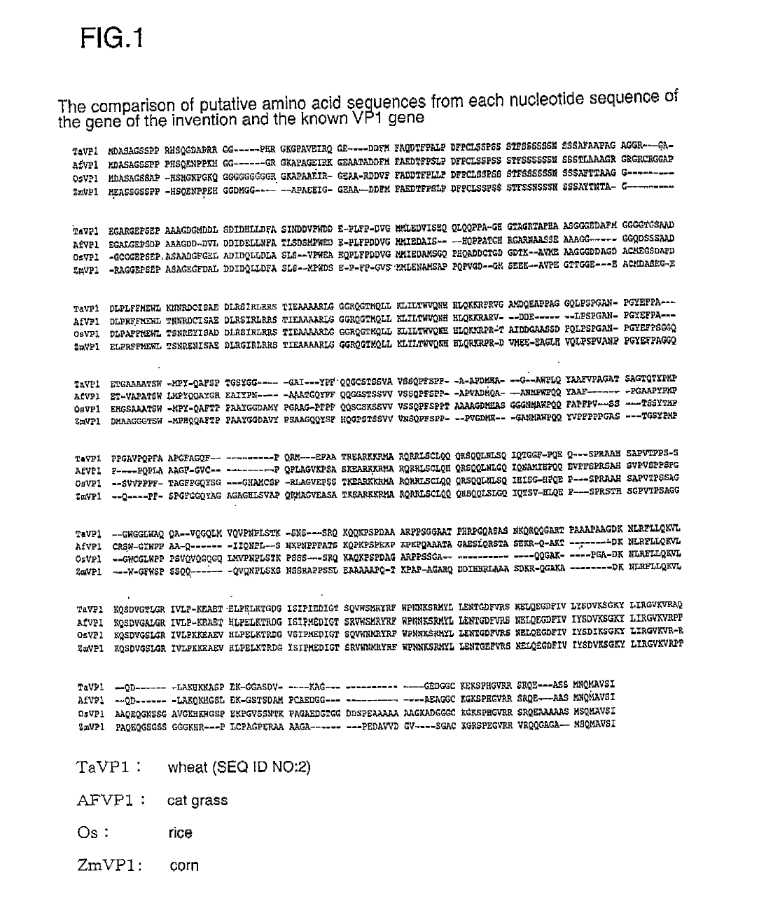

Furthermore, the obtained amino acid sequence (SEQ ID NO: 2) was compared with a putative amino acid sequence from a nucleotide sequence of VP1 gene which is known in relation to different types of plants (cat grass, rice and field corn). The results are shown in FIG. 1.

From the results indicated in FIG. 1, the amino acid sequence (SEQ ID NO: 2) encoded by a gene of the invention was confirmed to show 66.6%, 60.5%, 58.6% homology to the sequences encoded by VP1 gene of cat grass, that of rice, and that of field corn, respectively. These values are extremely high, which indicates that they were encoded by homologous genes although their sequences differs to some extent.

### EXAMPLE 4

Elucidation of the Correlation Between the Expression Level and Seed Dormancy in a Seed Embryo of a Gene Presumed to be Wheat VP 1 Gene.

Whole RNA was isolated form wheat seed samples of the same varieties (Zenkoujikomugi, Minaminokomugi, Sanin 1 and Tozan 18) as used in Example 1 and 2 using Trizole agent (Gibco Lifetech Oriental, USA). Then, only mRNA was extracted from the whole RNA using the Latex beads kit (TAKARA). These mRNA were run on agarose gel electrophoresis respectively, followed by blotting to N plus membrane (Amersham pharmacia, Sweden). DNA of a gene of the invention obtained above was then labeled with Alfos Direct Labelling Detection Kit (Amersham Pharmacia, Sweden) to be used as a probe, thereby performing hybridization to mRNA from samples of each variety blotted on the membrane.

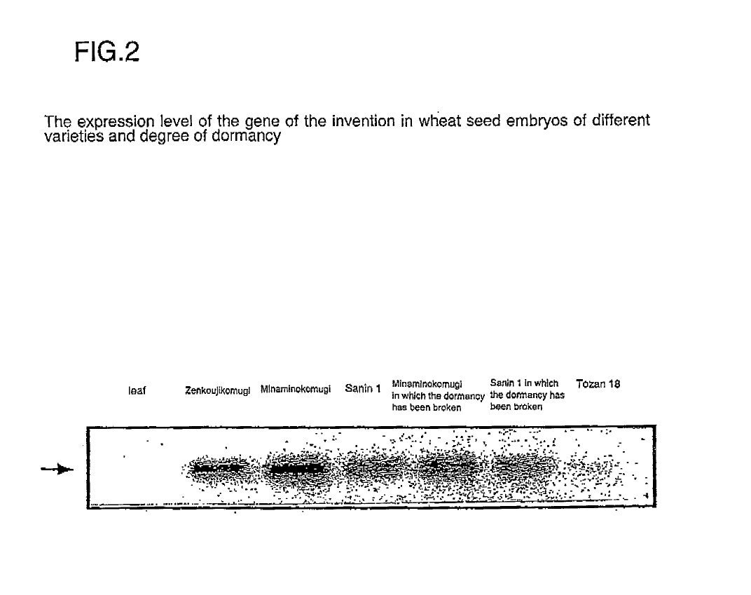

The expression level of a gene of the invention was determined by judging the degree of hybridization to DNA of the gene of the invention used as a probe from the strength of a detected label. The results are shown in FIG. 2.

As a result the correlation was confirmed between the degree of dormancy(strong/weak) and the expression level of said gene. That is to say, the expression level of said gene was high in a seed embryo of varieties with strong dormancy, “Minaminokomugi” and “Zenkoujikomugi” while the expression level of said gene was low in a seed embryo of varieties with weak dormancy, “Sanin 1” and “Tozan 18”, and in seed embryos in which dormancy has been disrupted, “Minaminokomugi” and “Sanin 1”.

Considering the above results and the results obtained from Example 1 and 2 together, said gene presumed to be wheat VP1 gene is considered to alter the degree of abscisic acid-sensitivity of a seed depending on the difference of the expression level between varieties, thereby changing the degree of dormancy of said seed.

### Effect of the Invention

The present invention provides a gene which regulates the dormancy of wheat seed, an antisense DNA to said gene, and a protein having activity which alters the degree of dormancy of wheat seed.

### Free Text of Sequence Listing

SEQ ID NO: 2: a putative amino acid sequence from a nucleotide sequence of SEQ ID NO: 1.

SEQ ID NO: 3: a forward primer used to amplify a sequence of the gene of the invention

SEQ ID NO: 4: a reverse primer used to amplify a sequence of the gene of the invention.

All publications, oatents and patent applications cited herein are incorporated herein by reference in their entirety.

1. An isolated gene encoding the amino acid sequence of SEQ ID NO: 2.

2. An antisense DNA to the gene according to claim 1.

3. An isolated protein comprising the amino acid sequence of SEQ ID NO: 2.

4. A recombinant vector comprising the gene according to claim 1.

5. A recombinant vector comprising the antisense DNA according to claim 2.

6. A transformant comprising the recombinant vector according to claim 4.

7. A transformant comprising the recombinant vector according to claim 5.

8. A method for producing a protein having activity which alters the degree of dormancy of wheat seed comprising culturing the transformant according claim 6 and recovering the protein from the resultant culture.

9. A method for producing a protein having activity which alters the degree of dormancy of wheat seed comprising culturing the transformant according claim 7 and recovering the protein from the resultant culture.
